(19) United States
(12) Patent Application Publication
(10) Pub. No.: US 2006/0021085 A1
(43) Pub. Date: Jan. 26, 2006

(54) Method for producing transgenic plants having an elevated vitamin E content by modifying the serine-acetyltransferase content

(75) Inventors: Michael Geiger, Quedlinburg (DE); Susanne Tropf, Quedlinburg (DE); Klaus-Dieter Salchert, Gernrode (DE); Ulrich Keetman, Quedlinburg (DE); Karin Herbers, Quedlinburg (DE); Rainer Lemke, Quedlinburg (DE); Rudiger Hell, Mannheim (DE); Markus Wirtz, Heidelberg (DE)

Correspondence Address: CONNOLLY BOVE LODGE & HUTZ, LLP, P O BOX 2207, WILMINGTON, DE, 19899, US

(73) Assignees: SunGene GmbH & Co. KGaA, Gatersleben (DE); IPK-Institut Fur Pflanzengenetik Und Kulturpflanzenforschung, Gatersleben (DE)

(21) Appl. No.: 11/170,711

(22) Filed: Jun. 23, 2005

Related U.S. Application Data
(63) Continuation of application No. PCT/EP03/14409, filed on Dec. 17, 2003.

(30) Foreign Application Priority Data
Dec. 23, 2002 (DE) 10260871.7

(51) Int. Cl. A01H 1/00; C12N 9/10; A01H 5/00; C12N 15/82; C12N 5/04

(52) U.S. Cl. 800/278; 435/468; 435/419; 435/193; 800/312; 800/320.1

(57) ABSTRACT

The invention relates to a method for producing transgenic plants and/or plant cells having an elevated vitamin E content, said transgenic plants and/or plant cells having a serine-acetyltransferase (SAT) content and/or activity which is modified in relation to the wild type, and/or a modified thiol compound content. The invention also relates to the use of nucleic acids coding for a SAT, for producing transgenic plants or plant cells having an elevated vitamin E content. The invention further relates to a method for producing vitamin E by cultivating transgenic plants or plant cells having a modified SAT content in relation to the wild type.

The present invention relates to a method for producing transgenic plants and/or plant cells having an increased vitamin E content, wherein the transgenic plants and plant cells, respectively, have an altered content and/or an altered activity of serine acetyltransferase (SAT) and/or an altered content of thiol compounds in comparison with the wild type. The present invention also relates to the use of nucleic acids coding for an SAT for producing transgenic plants and plant cells, respectively, having an increased vitamin E content. The present invention also relates to a method for producing vitamin E by cultivating transgenic plants and plant cells, respectively, having an SAT content altered in comparison with the wild type.

The eight naturally occurring compounds having vitamin E activity, which are derivatives of 6-chromanol, are usually referred to as vitamin E (Ullmann's Encyclopedia of Industrial Chemistry, Vol. A 27 (1996), VCH Verlagsgesellschaft, Chapter 4., 478-488, Vitamin E). The group of tocopherols (1) has a saturated side chain; the group of tocotrienols (2) has an unsaturated side chain. 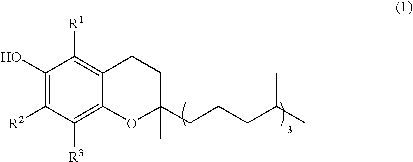
α-tocopherol: R¹ ═R²═R³═CH₃
β-tocopherol: R¹═R³═CH₃, R²═H
γ-tocopherol: R¹═H, R²═R³═CH₃
δ-tocopherol: R¹═R²═H, R³═CH₃ 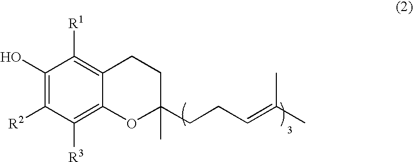
α-tocotrienol: R¹═R²═R³═CH₃
β-tocotrienol: R¹═R³═CH₃, R²═H
γ-tocotrienol: R¹═H, R²═R³═CH₃
δ-tocotrienol: R¹═R²═H, R³═CH₃

In the present invention, all above-mentioned tocopherols and tocotrienols having vitamin E activity are understood by vitamin E.

Said compounds having vitamin E activity are important natural fat-soluble antioxidants. Vitamin E deficiency leads to pathophysiological situations in humans and animals. Therefore, vitamin E compounds are of high economic value as additives in the fields of food and feed, in pharmaceutical formulations, and in cosmetic applications.

Of the above-mentioned compounds having vitamin E activity, α-tocopherol is biologically the most important. The tocopherols and tocotrienols occur in many vegetable oils; especially rich in tocopherols and tocotrienols are the seed oils of soy, wheat, maize, rice, cotton, rapeseed, lucerne and nuts. Fruits and vegetables, like e.g. raspberries, beans, peas, fennel, pepper etc., also contain the above-mentioned vitamin E compounds. As far as hitherto known, tocopherols and tocotrienols are synthesized exclusively in plants and photosynthetically active organisms, respectively. Some of the most important pathways of synthesis of tocopherols and tocotrienols are shown in FIGS. 1a and 1b.

Due to their redox potential, tocopherols contribute to the prevention of oxidation of unsaturated fatty acids by air-contained oxygen; in humans, α-tocopherol is the most important fat-soluble antioxidant. It is assumed that the tocopherols functioning as antioxidants contribute to the stabilization of biological membranes since membrane fluidity is maintained by protecting the unsaturated fatty acids of the membranes. Furthermore, according to recent findings, the formation of arteriosclerosis can be counteracted by regular administration of relatively high dosages of tocopherol. Further advantageous features of tocopherols were described to be the procrastination of diabetes-caused late damages, the reduction of the risk of cataract formation, the reduction of oxidative stress in smokers, anticarcinogenic effects, protective effect against skin damages like erythemae and skin aging. In this connection, tocopherol compounds like tocopherol acetate and succinate are the usual forms of application for the use of vitamin E in blood supply promoting and lipid lowering preparations and as feed additive in veterinary medicine.

Due to their oxidation-inhibiting properties, tocopherols and tocotrienols are not only utilized in food technology, but also in paints based on natural oils, in deodorants and other cosmetics, e.g. sun screening preparations, skin care preparations, lipsticks, etc.

Therefore, economical methods for the production of vitamin E compounds and food and feed having an increased vitamin E content, respectively, are of significant importance. In this connection, biotechnological methods or vitamin-E-producing organisms optimized by genetic engineering, like transgenic plants and plant cells, are particularly advantageous.

According to the prior art, enzymes involved in the biosynthesis of tocopherols and tocotrienols in higher plants are normally used for producing transgenic plants and plant cells, respectively, having an increased vitamin E content (see also FIGS. 1a and 1b).

In higher plants, tyrosine is formed starting from chorismate via prephenate and arogenate. The aromatic amino acid tyrosine is converted into hydroxy phenyl pyruvate by the enzyme tyrosine amino transferase, which is converted into homogentisic acid by dioxygenation.

The homogentisic acid is subsequently bound to phytyl pyrophosphate (PPP) and geranylgeranyl pyrophosphate, respectively, in order to form the α-tocopherol and α-tocotrienol precursors 2-methyl-6-phytyl-hydroquinone and 2-methyl-6-geranyl-geranyl-hydroquinone, respectively. Via methylation steps with S-adenosyl-methionine as methyl group donor, first 2,3-dimethyl-6-phytylquinone, then via cyclization γ-tocopherol and then via repeated methylation α-tocopherol are generated.

Attempts are known to achieve an increase in metabolite flow in order to increase the tocopherol and tocotrienol content, respectively, by overexpression of individual biosynthesis genes in transgenic organisms.

WO 97/27285 describes a modification of the tocopherol content by enhanced expression or by down-regulation of the enzyme p-hydroxyphenylpyruvate dioxygenase (HPPD).

WO 99/04622 and DellaPenna et al., (1998) Science 282, 2098-2100 describe gene sequences coding for a γ-tocopherol methyl transferase from Synechocystis PCC6803 and Arabidopsis thaliana and its insertion into transgenic plants having a modified vitamin E content.

WO 99/23231 shows that the expression of a geranylgeranyl reductase in transgenic plants leads to an enhanced tocopherol biosynthesis.

WO 00/08169 describes gene sequences encoding a 1-deoxy-D-xylose-5-phosphate-synthase and a geranylgeranyl pyrophosphate oxidoreductase and their insertion into transgenic plants having a modified vitamin E content.

WO 00/68393 and WO 00/63391 describe gene sequences encoding a phytyl/prenyl transferase and their insertion into transgenic plants having a modified vitamin E content.

In WO 00/61771 it is postulated that the combination of a gene from the sterol metabolism with a gene from the tocopherol metabolism can lead to an increase of the tocopherol content in transgenic plants.

While all these methods yield genetically engineered organisms, in particular plants, which usually have a modified vitamin E content, they have the disadvantage that the level of vitamin E content in the genetically engineered organisms known in the prior art is not yet satisfactory.

Therefore, there still is a great need for transgenic plants and plant cells, respectively, having significantly increased vitamin E contents, which can be utilized for obtaining the vitamin E compounds.

Therefore, the problem underlying the invention is to provide a method allowing the production of transgenic plants and plant cells, respectively, having an increased vitamin E content.

This and further problems underlying the invention, as resulting from the description, are solved by the subject matter of the independent claim.

Preferred embodiments of the invention are defined by the dependent subclaims.

It has now surprisingly been found that the alteration of the content and/or the activity of SAT in transgenic plants and plant cells, respectively, allows an increase of the content of vitamin E compounds like the above-mentioned tocopherols and tocotrienols. This was surprising in particular because it was hitherto assumed that enzymes having an SAT activity have a function only in pathways of biosynthesis for producing sulfurous compounds like cysteine, methionine and e.g. glutathione.

Serine acetyltransferase (SAT, EC2.3.1.30) is involved in the two-step process by which cysteine biosynthesis is accomplished in vivo in microorganisms and plants.

SAT provides the formation of the activated thioester O-acetylserine (OAS) from serine and acetyl coenzyme A. Free sulfide is incorporated into O-acetylserine in order to obtain cysteine and acetate by means of enzymatic catalysis via O-acetylserine (thiol)-lyase. In this connection, the reaction catalyzed by SAT represents the pace-limiting step, the activity of this enzyme being exclusively found in connection with O-acetylserine (thiol)-lyase (OAS-TL) in the so-called cysteine synthase complex. OAS-TL is available in great abundance due to the activity of SAT-free homodimers (Kredich et al., (1969) J. Biol. Chem., 244, 2428-2439; Saito (2000) Curr. Opin. Biol. 3, 188-195).

Microbial, plant and animal SATs can be subdivided into different groups according to their allosteric adjustability. Several SATs are inhibited by cysteine, the end product of the pathway of biosynthesis catalyzed by them. Such SATs are usually referred to as feedback-regulated SATs (Wirtz et al., (2002), Amino acids, in press; Hell et al., (2002) Amino Acids 22, 245-257; Noji et al., (1998) J. Biol. Chem. 273, 32739-45; Inoue et al., (1999) Eur. J. Biochem. 266, 220-27; Saito (2000) Curr. Op. Plant Biol. 3, 188-95).

The microbial SATs CysE from E. coli (Accession Code E12533; Denk and Bock (1987) J. Gen. Microbiol. 133, 515-25) and S. typhimurium (Accession Code A00198; Kredich and Tomkins (1966) J. Biol. Chem. 241, 4955-65), as well as the plant SATs SAT-c from A. thaliana (Accession Code U30298; Noji et al., vide supra), SAT2 from Citrullus vulgaris (Accession Code D49535; Saito et al., (1995) J. Biol. Chem. 270, 16321-26) and SAT56 from Spinacia oleracea (Accession Code D88529; Noji et al., (2001) Plant Cell Physiol. 42, 627-34) are regarded as prototypes for feedback-regulated SATs.

Besides, there is a group of SATs, which can be inhibited by cysteine to a substantially lesser extent or cannot be inhibited by cysteine at all, respectively. These SATs are also called feedback-independent SATs. Typical representatives of such feedback-independent SATs are hitherto only known from plants. Among these are e.g. Arabidopsis thaliana (EMBL Accession code X82888; Bogdanova and Hell (1995) Plant Physiol. 109, 1498; Wirtz et al., 2002, vide supra), SAT 4 from Nicotiana tabacum (Accession Code AJ414052; Wirtz et al., 2002, vide supra) and ASAT5 from Allium tuberosum (Urano et al., (2000) Gene 257, 269-277).

Due to their role in the pathway of biosynthesis for cysteine, the use of SAT-coding nucleic acid sequences for producing transgenic plants and plant cells, respectively, has been discussed only in connection with methods for producing transgenic organisms having increased cysteine and glutathione contents, respectively (Wirtz et al., vide supra). No functional connection between SAT and pathways of biosynthesis leading to the production of vitamin E compounds like tocopherols and tocotrienols is known from the prior art.

Within the scope of the present invention it has now surprisingly been found that the alteration of the content or the activity of functional or non-functional SATs in plants can be used for producing transgenic plants and plant cells, respectively, having increased vitamin E contents. The alteration of the content and/or the activity of SATs in transgenic plants and plant cells, respectively, can, in this connection, be due to e.g. the transfer and overexpression of nucleic acids coding for functional or non-functional SATs, to plant cells and plants, respectively. The alteration of the content and/or the activity of SAT in transgenic plants and plant cells, respectively, having an increased vitamin E content can also be due to the up- or down-regulation, respectively, of the activity and/or of the synthesized amounts of endogenous SATs.

Furthermore, it has now been found within the scope of the present invention that the alteration of the content of thiol compounds can be used for producing transgenic plants and plant cells, respectively, having increased vitamin E contents. The alteration of the content of thiol compounds can, in this connection, be due to e.g. the alteration of the content and/or the activity of SATs in transgenic plants and plant cells, respectively, and can therefore be achieved, e.g., by transfer and overexpression of nucleic acids coding for functional or non-functional SATs to plant cells and plants, respectively. In this connection, however, the alteration of the content of thiol compounds can also be due to the alteration of the content and/or the activity of other enzymes involved in the metabolic pathways of thiol compounds and can therefore be achieved, e.g., by the transfer and overexpression of nucleic acids coding for such enzymes or homologues, mutants and fragments thereof, respectively, to plant cells and plants, respectively.

Object of the present invention is therefore a method for producing transgenic plants and plant cells, respectively, having an increased vitamin E content and having an altered content and/or an altered activity of SAT in comparison with the wild type.

Likewise, object of the invention is a method for producing transgenic plants and plant cells, respectively, having an increased vitamin E content, wherein the expression of SAT is caused by transfer of nucleic acid sequences coding for functional or non-functional SATs or functional equivalents thereof to plants and plant cells, respectively.

Further objects of the present invention are methods for producing transgenic plants and plant cells, respectively, having an increased vitamin E content, wherein the activity or the amount of endogenous SAT is up- or down-regulated.

A further object of the invention is a method for producing transgenic plants and plant cells, respectively, having an increased vitamin E content, wherein antibodies specific for SATs and possibly inhibiting their function are expressed in the cell.

Further objects of the invention are methods for producing transgenic plants and plant cells, respectively, having an increased vitamin E content, wherein the post-translational modification state of overexpressed or endogenous functional or non-functional SATs is altered.

Likewise, objects of the present invention are methods for producing transgenic plants and plant cells, respectively, having an increased vitamin E content, wherein the expression of a part of the endogenous SAT genes was silenced by means of methods like e.g. antisense methods, post transcriptional gene silencing (PTGS), virus-induced gene silencing (VIGS), RNA interference (RNAi) or homologous recombination.

Objects of the invention are also transgenic plants and plant cells, respectively, having an increased vitamin E content, which have an altered content and/or an altered activity of SAT in comparison with the wild type.

Object of the present invention is also a method for producing transgenic plants and plant cells, respectively, having an increased vitamin E content, wherein the plants have an altered content of thiol compounds in comparison with the wild type.

Objects of the present invention are also transgenic plants and plant cells, respectively, produced according to a method according to the present invention and having increased vitamin E contents in comparison with the wild type.

A further object of the present invention is the use of nucleic acids coding for functional or non-functional SATs from different organisms for producing transgenic plants and plant cells, respectively, having an increased vitamin E content.

According to the present invention, serine acetyltransferase activity is understood to be the enzymatic activity of a serine acetyltransferase.

A serine acetyltransferase is understood to be a protein having the enzymatic activity to link serine and acetyl coenzyme A to form the activated thioester O-acetylserine (OAS).

Accordingly, serine acetyltransferase activity is understood to be the amount of serine converted or the amount of O-acetylserine formed, respectively, by the protein serine acetyltransferase during a certain time.

According to the present invention, in the case of an SAT activity altered in comparison with the wild type, a different amount of serine is converted or a different amount of O-acetylserine is formed, respectively, during a certain time by the protein SAT.

Therefore, according to the present invention, in the case of an SAT activity increased in comparison with the wild type, the converted amount of serine or the formed amount of O-acetylserine, respectively, is increased by the protein SAT during a certain time in comparison with the wild type.

Therefore, in the case of an SAT activity decreased in comparison with the wild type, the converted amount of serine and the formed amount of O-acetylserine, respectively, is decreased by the protein SAT during a certain time.

Therefore, in the case of an SAT content altered in comparison with the wild type, a different amount of the protein SAT is produced in the plant and plant cell, respectively, in comparison with the wild type.

Therefore, in the case of an SAT content increased in comparison with the wild type, more SAT is produced in the plant and plant cell, respectively, in comparison with the wild type.

Accordingly, in the case of an SAT content decreased in comparison with the wild type, less SAT is produced in the plant and plant cell, respectively, in comparison with the wild type.

Therefore, in the case of an altered content of thiol compounds in comparison with the wild type, a different amount of thiol compounds is produced in the plant and plant cell, respectively, in comparison with the wild type. The equivalent applies to increased and decreased thiol contents, respectively.

Preferably, the increase of the content and/or the activity of SAT, which is caused by a method according to the present invention, in a transgenic plant cell and plant, respectively, amounts to at least 5%, preferably at least 20%, also preferably at least 50%, particularly preferred at least 100%, also particularly preferred at least the factor 5, particularly preferred at least the factor 10, also particularly preferred at least the factor 50, more preferably at least the factor 100 and most preferably at least the factor 1000.

Preferably, the decrease of the content and/or the activity of SAT, which is caused by a method according to the present invention, in a transgenic plant cell and plant, respectively, amounts to at least 5%, preferably at least 10%, particularly preferred at least 20%, also particularly preferred at least 40%, also particularly preferred at least 60%, in particular preferred at least 80%, also in particular preferred at least 90% and most preferably at least 98%.

According to the present invention, a wild type is understood to be the corresponding original organism, which is not genetically engineered.

When SAT is mentioned within the scope of the present invention, both feedback-regulated and feedback-independent SATs are meant according to the present invention. Within the scope of the present invention, the term SAT comprises functional and non-functional SATs.

In this connection, functional SATs fall within the definition of an SAT as given above.

When functionally equivalent parts of SATs are mentioned within the scope of the present invention, fragments of nucleic acid sequences of complete SATs are meant, whose expression still leads to proteins having the enzymatic activity of an SAT. These protein fragments also fall within the term “functionally equivalent parts of SATs”.

According to the present invention, non-functional SATs have the same nucleic acid sequences and amino acid sequences, respectively, as functional SATs and functionally equivalent parts thereof, respectively, but have, at some positions, point mutations, insertions or deletions of nucleotides or amino acids, which have the effect that the non-functional SATs are not, or only to a very limited extent, capable of acetylating serine while forming O-acetylserine. Non-functional SATs also comprise such SATs bearing point mutations, insertions, or deletions at the nucleic acid sequence level or amino acid sequence level and are not, or nevertheless, capable of interacting with physiological binding partners of SAT. Such physiological binding partners comprise, e.g. O-acetylserine (thiol)-lyase.

According to the present invention, the term “non-functional SAT” does not comprise such proteins having no essential sequence homology to functional SATs at the amino acid level and nucleic acid level, respectively. Proteins unable to transfer acetyl groups to serine and having no essential sequence homology with SATs are therefore, by definition, not meant by the term “non-functional SATs” of the present invention. Non-functional SATs are, within the scope of the present invention, also referred to as inactivated or inactive SATs.

Therefore, non-functional SATs according to the present invention bearing the above-mentioned point mutations, insertions, and/or deletions are characterized by an essential sequence homology to the known functional SATs according to the present invention or functionally equivalent parts thereof.

According to the present invention, a substantial sequence homology is generally understood to indicate that the nucleic acid sequence or the amino acid sequence, respectively, of a DNA molecule or a protein, respectively, is at least 40%, preferably at least 50%, further preferred at least 60%, also preferably at least 70%, particularly preferred at least 90%, in particular preferred at least 95% and most preferably at least 98% identical with the nucleic acid sequences or the amino acid sequences, respectively, of a known functional SAT or functionally equivalent parts thereof.

Identity of two proteins is understood to be the identity of the amino acids over the respective entire length of the protein, in particular the identity calculated by comparison with the assistance of the Lasergene software by DNA Star, Inc., Madison, Wis. (USA) applying the CLUSTAL method (Higgins et al., (1989), Comput. Appl. Biosci., 5(2), 151).

Homologies can also be calculated with the assistance of the Lasergene software by DNA Star, Inc., Madison, Wis. (USA) applying the CLUSTAL method (Higgins et al., (1989), Comput. Appl. Biosci., 5(2), 151).

Nucleic acid sequences or amino acid sequences of feedback-regulated or feedback-independent functional SATs are known to the person skilled in the art. They can e.g. be taken from the generally known databases like the nucleotide sequence database GenBank or the protein sequence database of the NCBI. Furthermore, numerous examples for said SATs can be found in the literature (see above).

Particularly preferred for the methods according to the present invention are the nucleic acid sequences for feedback-regulated functional SATs from A. thaliana (SAT-c; U30298; Noji et al., (1998) vide supra), from Citrullus vulgaris (SAT2; Accession Code D49535; Saito et al., (1995) J. Biol. Chem. 270, 16321-26) and from Spinacia oleracea (SAT56; D88529; Noji et al., (2001) Plant Cell Physiol. 42, 627-34) as well as from microorganisms like S. typhimurium (CysE; Accession Code A00198; Kredich and Tomkins (1966) J. Biol. Chem. 241, 4955-65).

Likewise, particularly preferred for the methods according to the present invention are the nucleic acid sequences for feedback-independent functional SATs from plants, microorganisms, fungi, and animals. In this connection, the cDNA sequences of Nicotiana tabacum SAT-genes 1, 4 and 7 (EMBO Accession numbers AJ414051, AJ414052 and AJ414053) as well as the Arabidopsis thaliana SAT-genes SAT 52, SAT 5 and SAT A (EMBL Accession Codes U30298, Z34888 und X82888) are particularly preferred.

Further preferred nucleic acid sequences for said SATs comprise the A. thaliana genes SAT-p (Accession Code L42212; Noji et al., (1998) vide supra) and SAT-m (identical with SAT A; Accession code X82888; Noji et al., (1998) vide supra; Bogdanova and Hell (1995) Plant Physiol., 109, 1498; Wirtz et al., (2002) vide supra).

SATs with sequences being substantially homologous to the sequences of the above-mentioned accession numbers are also objects of preferred embodiments of the invention.

Particularly preferred for the use in the method according to the present invention are nucleic acids encoding proteins, comprising the amino acid sequence GKXXGDRHPKIGD (X being an arbitrary amino acid; Wirtz et al., (2001) Eur. J. Biochem. 268, 686-93) or a sequence derived from said sequence by substitution, insertion or deletion of amino acids, which has an identity of at least 30%, preferably of at least 50%, preferably of at least 70%, more preferably of at least 90%, most preferably of at least 95% at the amino acid level with the sequence having the accession code X82888 (Bogdanova et al., (1995) FEBS L. 358, 43-47; Bogdanova and Hell (1995) Plant Physiol. 109, 1498; Wirtz et al., 2002, vide supra) and having the enzymatic activity of an SAT.

Non-functional feedback-regulated or non-functional feedback-independent SATs according to the present invention can easily be identified by the person skilled in the art. The person skilled in the art has at his disposal several techniques, with which it is possible to introduce mutations, insertions or deletions into the nucleic acid sequences coding for functional SATs (Sambrook (2001), Molecular Cloning: A Laboratory Manual, 3rd Edition, Cold Spring Harbor Laboratory Press). After introducing the point mutation, insertion and/or deletion, which are generally referred to as mutation, the person skilled in the art can, by means of corresponding enzyme activity tests as depicted in the Examples or as known from the prior art, ascertain if the mutagenized SATs still have enzymatic activity. Non-functional SATs have a decreased activity compared to non-mutagenized SAT. According to the present invention, a non-functional SAT has 1 to 90%, preferably 1 to 70%, particularly preferred 1 to 50%, also particularly preferred 1 to 30%, in particular preferred 1 to 15% and most preferably 1 to 10% of the activity of the corresponding functional SAT having a wild type sequence.

The person skilled in the art can also identify non-functional SATs, which are not capable anymore (or nevertheless capable) of binding to physiological binding partners of the SAT, like e.g. OAS-TL, in routine experiments by means of corresponding in vitro binding tests.

Preferably, nucleic acid sequences coding for a non-functional SAT having reduced enzymatic activity are used as non-functional SATs for the methods according to the present invention, wherein the SAT has at least one amino acid substitution within the amino acid motif
GKX₁X₂GDRHPKIGD
which is conserved in SAT enzymes. The amino acid X is generally an arbitrary amino acid, X₁ is preferably Q or A; the amino acid X₂ is preferably C or S. Amino acids are abbreviated using the one-letter-code. The amino acids located N— and C-terminally, respectively, next to said motif are strongly conserved in SATs. The core motif within said amino acid sequence motif is DRH. An amino acid substitution within this core motif is particularly preferred.

In a particularly preferred embodiment, the mutation leading to the enzymatic inactivation of the SAT is an amino acid substitution of the amino acid histidine within said motif. Here, a substitution of histidine with alanine is particularly preferred.

The term “point mutation” in the description is to be understood as the substitution of an amino acid or a nucleotide with another amino acid or another nucleotide. Concerning amino acids, so-called conservative substitutions are preferably performed, wherein the substituted amino acid has physico-chemical properties similar to those of the original amino acid, e.g. a substitution of glutamate with aspartate or valine with isoleucine. Deletion is the substitution of an amino acid or of a nucleotide with a direct bond. Insertions are introductions of amino acids or nucleotides into the polypeptide chain or into the nucleic acid molecule, wherein a direct bond is formally substituted with one or more amino acids or nucleotides.

Different SAT amino acid sequences are comparatively shown in the appended FIG. 2. Herein, SAT1 stands for the Arabidopsis thaliana SAT isoform A (SAT-1, database axcession no. U 22964), SAT5 stands for the Arabidopsis thaliana SAT isoform B (SAT-5, database axcession no. Z 34888), SAT52 stands for the Arabidopsis thaliana SAT isoform C (SAT-52, database axcession no. U 30298), CysE stands for the SAT enzyme from S. typhimurium (CysE, database accession no. A 00198); TDT stands for tetrahydrodipicolinate-N-succinyltransferase from E. coli (TDT; database accession no. P 56220); LpxA stands for UDP-N-acetylglucosamin-acyltransferase from E. coli (LpxA; database accession no. P 10440). Further information concerning sequences is to be found in Murillo et al., (1995) Cell. Mol. Biol. Res. 41, 425-433; Howarth et al., (1997) Biochim. Biophys. Acta 1350, 123-127; Saito et al., (1995) J. Biol. Chem. 270, 16321-16326, GenBank Accession no. D 88530 (K. Saito). The position of the motif suitable for the inactivation of the SAT enzyme can be taken from the appended alignment. Correspondingly, the position of the conserved amino acid motif can be determined by alignments in further SAT enzymes, which are to be taken from the prior art. For example, the core motif D R H in the Arabidopsis thaliana SAT isoform A is located at amino acids 307-309, wherein the numbering always refers to the first methionine of the longest open reading frame. The position of the motif in the other SAT isoforms can easily be taken from the amino acid alignment, which is appended as FIG. 2.

Beside the above-mentioned SAT genes, the person skilled in the art has at his disposal further SAT sequences described in the prior art and available from gene databases, which are suitable for the realization of the invention. Furthermore, the person skilled in the art is capable of isolating further SAT gene sequences from a desired organism without any problems by using routine methods like PCR or screening of libraries with suitable SAT gene probes.

A multiplicity of DNA sequences coding for both functional and non-functional, feedback-regulated and feedback-independent SATs, respectively, from various organisms have already been given in the above. It is known to the person skilled in the art how to isolate corresponding DNA sequences from other organisms. Typically, the person skilled in the art will first try to identify corresponding homologous sequences by means of homology comparisons in established databases, like e.g. the GenBank database at the NCBI. Such databases can be found on the NCBI homepage at the NIH under http://www.ncbi.nlm.nih.gov.

DNA sequences having a high homology, i.e. a high similarity or identity are bona fide candidates for DNA sequences, which correspond to the DNA sequences according to the present invention, i.e. SATs. These gene sequences can be isolated by means of standard methods, like e.g. PCR and hybridization, and their function can be determined by means of suitable enzyme activity tests and other experiments by the person skilled in the art. Homology comparisons with DNA sequences can, according to the present invention, also be used for designing PCR primers by firstly identifying their regions, which are most conserved among the DNA sequences of different organisms. Such PCR primers can then be used for isolating, in a first step, DNA fragments, which are components of DNA sequences, which are homologous to the DNA sequences according to the invention.

There are a variety of search engines, which can be used for such homology comparisons and searches, respectively. These search engines comprise, e.g., the CLUSTAL program group of the BLAST program, which is provided by the NCBI.

Furthermore, a variety of experimental methods for isolating DNA sequences from most different organisms, which are homologous to the SATs according to the present invention, are known to the person skilled in the art. These comprise, e.g., the preparation and screening of cDNA libraries with correspondingly degenerated probes (see also Sambrook et al., vide supra).

Object of the present invention is also a method for producing transgenic plants and plant cells, respectively, having an increased vitamin E content, wherein the plants have an altered content of thiol compounds in comparison with the wild type. In a preferred embodiment of the invention, these transgenic plants have increased contents of thiol compounds in comparison with the wild type. In this connection, the increase of thiol compounds can amount to at least the factor 2, preferably at least the factor 5, particularly preferred at least the factor 10, in particular preferred at least the factor 20 and most preferably at least the factor 100. The increase of the vitamin E content usually corresponds to the values mentioned further below.

According to the present invention, thiol compounds are understood to be compounds naturally occurring in plants and having thiol groups. In particular, thiol compounds comprise glutathione, S-adenosylmethionine, methionine, and cysteine.

According to the present invention, transgenic plants with an altered content of thiol compounds can be produced by, e.g., altering the content and/or the activity of SAT as discussed in detail in the following. However, such transgenic plants can also be produced by altering the content and/or the activity of other enzymes, which are involved in the production of thiol compounds.

The increase of the SAT activity and the SAT content can be achieved via different routes, e.g. by switching off inhibitory regulatory mechanisms at the transcription, translation, and protein level or by increase of gene expression of a nucleic acid coding for an SAT in comparison with the wild type, e.g. by inducing the SAT gene or by introducing nucleic acids coding for an SAT.

In a preferred embodiment, the increase of the SAT activity and the SAT content, respectively, in comparison with the wild type is achieved by an increase of the gene expression of a nucleic acid encoding an SAT. In a further preferred embodiment, the increase of the gene expression of a nucleic acid encoding an SAT is achieved by introducing nucleic acids encoding an SAT into the organism, preferably into a plant.

In principle, every SAT gene of different organisms, i.e. every nucleic acid encoding an SAT, can be used here. With genomic SAT nucleic acid sequences from eukaryotic sources containing introns, already processed nucleic acid sequences like the corresponding cDNAs are to be used in the case that the host organism is not capable or cannot be made capable of splicing the corresponding SATs. All nucleic acids mentioned in the description can be, e.g., an RNA, DNA or cDNA sequence.

In a preferred method according to the present invention for producing transgenic plants and plant cells, respectively, having an increased vitamin E content, a nucleic acid sequence coding for one of the above-defined functional or non-functional, feedback-regulated or feedback-independent SATs, is transferred to a plant and plant cell, respectively. This transfer leads to an increase of the expression of the functional and non-functional SAT, respectively, and correspondingly to an increase of the vitamin E content in the transgenic plants and plant cells, respectively.

According to the present invention, such a method typically comprises the following steps:
a) production of a vector comprising the following nucleic acid sequences, preferably DNA sequences, in 5′-3′-orientation:
   a promoter sequence functional in plants
   operatively linked thereto a DNA sequence coding for an SAT or functional equivalent parts thereof
   a termination sequence functional in plants
b) transfer of the vector from step a) to a plant cell and, optionally, integration into the plant genome.

Such a method can be used for increasing the expression of DNA sequences coding for functional or non-functional, feedback-regulated or feedback-independent SATs or functionally equivalent parts thereof and therefore also increasing the vitamin E content in plants and plant cells, respectively. The use of such vectors comprising regulatory sequences, like promoter and termination sequences are, is known to the person skilled in the art. Furthermore, the person skilled in the art knows how a vector from step a) can be transferred to plant cells and which properties a vector must have to be able to be integrated into the plant genome.

By overexpression of active SAT, the total activity of SAT can, in this way, be increased by up to the factor 100 in leaves of transgenic tobacco (Wirtz et al. (2002) vide supra). The measurable SAT total activity does correspondingly not increase after overexpression of non-functional SAT, but the amount of non-functional SAT does increase. Nevertheless, the formation rate of O-acetylserine and cysteine must have increased in these transgenic lines, as the contents of these compounds strongly increase (WO 02/060939). Without intending to be bound by a scientific hypothesis, it is assumed that this increase is most likely achieved by compensation of the respective cell compartments that are not affected, which have their own cysteine synthesis enzymes, in order to compensate the deregulation in plastids and the cytosol, respectively, caused by inactivated SAT (WO 02/060939). Simultaneously, a significant increase of the vitamin E content in the plants is achieved.

Generally, an increase of the vitamin E content of at least 20%, preferably at least 50%, also preferably at least 75%, particularly preferred at least 100%, in particular preferred by at least the factor 5, also particularly preferred at least the factor 10 and most preferably at least the factor 100 in comparison with the wild type can be achieved by means of the depicted method.

If the SAT content in transgenic plants and plant cells, respectively, is increased by transferring a nucleic acid coding for an SAT from another organism, like e.g. E. coli, it is advisable to transfer the amino acid sequence encoded by the nucleic acid sequence e.g. from E. coli by back-translation of the polypeptide sequence according to the genetic code into a nucleic acid sequence comprising mainly those codons, which are used more often due to the organism-specific codon usage. The codon usage can be determined by means of computer evaluations of other known genes of the relevant organisms.

According to the present invention, an increase of the gene expression and of the activity, respectively, of a nucleic acid encoding an SAT is also understood to be the manipulation of the expression of the endogenous SATs of an organism, in particular of a plant. This can be achieved, e.g., by altering the promoter DNA sequence for genes encoding SAT. Such an alteration, which causes an altered, preferably increased, expression rate of at least one endogenous SAT gene, can be achieved by deletion or insertion of DNA sequences.

An alteration of the promoter sequence of endogenous SAT genes usually causes an alteration of the expressed amount of the SAT gene and therefore also an alteration of the SAT activity detectable in the cell or in the plant.

Furthermore, an altered and increased expression, respectively, of at least one endogenous SAT gene can be achieved by a regulatory protein, which does not occur in the transformed organism, and which interacts with the promoter of these genes. Such a regulator can be a chimeric protein consisting of a DNA binding domain and a transcription activator domain, as e.g. described in WO 96/06166.

A further possibility for increasing the activity and the content of endogenous SATs is to up-regulate transcription factors involved in the transcription of the endogenous SAT genes, e.g. by means of overexpression. The measures for overexpression of transcription factors are known to the person skilled in the art and are also disclosed for SATs within the scope of the present invention.

Furthermore, an alteration of the activity of endogenous SATs can be achieved by targeted mutagenesis of the endogenous gene copies.

An alteration of the endogenous SATs can also be achieved by influencing the post-translational modifications of SATs. This can happen e.g. by regulating the activity of enzymes like kinases or phosphatases involved in the post-translational modification of SATs by means of corresponding measures like overexpression or gene silencing.

The expression of endogenous SATs can also be regulated via the expression of aptamers specifically binding to the promoter sequences of SAT. Depending on the aptamers binding to stimulating or repressing promoter regions, the amount and thus, in this case, the activity of endogenous SAT is increased or reduced.

Aptamers can also be designed in a way as to specifically bind to the SAT proteins and reduce the activity of the SATs by e.g. binding to the catalytic center of the SATs. The expression of aptamers is usually achieved by vector-based overexpression and is, as well as the design and the selection of aptamers, well known to the person skilled in the art (Famulok et al., (1999) Curr Top Microbiol Immunol., 243,123-36).

Furthermore, a decrease of the amount and the activity of endogenous SATs can be achieved by means of various experimental measures, which are well known to the person skilled in the art. These measures are usually summarized under the term “gene silencing”. For example, the expression of an endogenous SAT gene can be silenced by transferring an above-mentioned vector, which has a DNA sequence coding for SAT or parts thereof in antisense order, to plants. This is based on the fact that the transcription of such a vector in the cell leads to an RNA, which can hybridize with the mRNA transcribed by the endogenous SAT gene and therefore prevents its translation.

In principle, the antisense strategy can be coupled with a ribozyme method. Ribozymes are catalytically active RNA sequences, which, if coupled to the antisense sequences, cleave the target sequences catalytically (Tanner et al., (1999) FEMS Microbiol Rev. 23 (3), 257-75). This can enhance the efficiency of an antisense strategy.

Further methods for reducing the SAT expression, in particular in plants as organisms, comprise the overexpression of homologous SAT nucleic acid sequences leading to co-suppression (Jorgensen et al., (1996) Plant Mol. Biol. 31 (5), 957-973) or inducing the specific RNA degradation by the plant with the aid of a viral expression system (Amplikon) (Angell et al., (1999) Plant J. 20 (3), 357-362). These methods are also referred to as “post-transcriptional gene silencing” (PTGS).

Further methods are the introduction of nonsense mutations into the endogenous gene by means of introducing RNA/DNA oligonucleotides into the plant (Zhu et al., (2000) Nat. Biotechnol. 18 (5), 555-558) or generating knockout mutants with the aid of e.g. T-DNA mutagenesis (Koncz et al., (1992) Plant Mol. Biol. 20 (5) 963-976) or homologous recombination (Hohn et al., (1999) Proc. Natl. Acad. Sci. USA. 96, 8321-8323.).

Furthermore, a gene repression (but also gene overexpression) is also possible by means of specific DNA-binding factors, e.g. factors of the zinc finger transcription factor type. Furthermore, factors inhibiting the target protein itself can be introduced into a cell. The protein-binding factors can e.g. be aptamers (Famulok et al., (1999) Curr Top Microbiol Immunol. 243, 123-36).

As further protein-binding factors, whose expression in plants causes a reduction of the content and/or the activity of SAT, SAT-specific antibodies may be considered. The production of monoclonal, polyclonal, or recombinant SAT-specific antibodies follows standard protocols (Guide to Protein Purification, Meth. Enzymol. 182, pp. 663-679 (1990), M. P. Deutscher, ed.). The expression of antibodies is also known from the literature (Fiedler et al., (1997) Immunotechnology 3, 205-216; Maynard and Georgiou (2000) Annu. Rev. Biomed. Eng. 2, 339-76).

Further techniques, which can be used to suppress, minimize or prevent the expression of endogenous SAT genes, comprise VIGS, RNAi or gene knockouts e.g. by means of homologous recombination. The corresponding methods are known to the person skilled in the art or can easily be searched in the literature. A further common method of gene silencing is co-suppression (see e.g. Waterhouse et al., (2001), Nature 411, 834-842; Tuschl (2002), Nat. Biotechnol. 20, 446-448 and further publications in this edition, Paddison et al., (2002), Genes Dev. 16, in press, Brummelkamp et al., (2002), Science 296, 550-553).

The mentioned techniques are well known to the person skilled in the art. Therefore, he also knows which sizes the nucleic acid constructs used for e.g. antisense methods or RNAi methods must have and which complementarity, homology or identity, the respective nucleic acid sequences must have.

The terms complementarity, homology, and identity are known to the person skilled in the art.

Within the scope of the present invention, sequence homology and homology, respectively, are generally understood to mean that the nucleic acid sequence or the amino acid sequence, respectively, of a DNA molecule or a protein, respectively, is at least 40%, preferably at least 50%, further preferred at least 60%, also preferably at least 70%, particularly preferred at least 90%, in particular preferred at least 95% and most preferably at least 98% identical with the nucleic acid sequences or amino acid sequences, respectively, of a known DNA or RNA molecule or protein, respectively. Herein, the degree of homology and identity, respectively, refers to the entire length of the coding sequence.

The term complementarity describes the capability of a nucleic acid molecule of hybridizing with another nucleic acid molecule due to hydrogen bonds between two complementary bases. The person skilled in the art knows that two nucleic acid molecules do not have to have a complementarity of 100% in order to be able to hybridize with each other. A nucleic acid sequence, which is to hybridize with another nucleic acid sequence, is preferred being at least 40%, at least 50%, at least 60%, preferably at least 70%, particularly preferred at least 80%, also particularly preferred at least 90%, in particular preferred at least 95% and most preferably at least 98 or 100%, respectively, complementary with said other nucleic acid sequence.

Nucleic acid molecules are identical, if they have identical nucleotides in identical 5′-3′-order.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., vide supra). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleotides). In addition, stringent conditions can comprise the addition of destabilizing agents like e.g. formamide. Typical hybridization and washing buffers are of the following composition.

| | |
|---|---|
| Pre-hybridization solution: | 0.5% SDS |
| | 5× SSC |
| | 50 mM NaPO₄, pH 6.8 |
| | 0.1% Na-pyrophosphate |
| | 5× Denhardt's reagent |
| | 100 μg/salmon sperm |
| Hybridization solution: | Pre-hybridization solution |
| | 1 × 10⁶ cpm/ml probe (5-10 min 95° C.) |
| 20× SSC: | 3 M NaCl |
| | 0.3 M sodium citrate |
| | ad pH 7 with HCl |
| 50× Denhardt's reagent: | 5 g Ficoll |
| | 5 g polyvinylpyrrolidone |
| | 5 g Bovine Serum Albumin |
| | ad 500 ml A. dest. |

A typical procedure for the hybridization is as follows:

| Optional: | wash Blot 30 min in 1× SSC/0.1% SDS at 65° C. | | |
|---|---|---|---|
| Pre-hybridization: | at least 2 h at 50-55° C. | | |
| Hybridization: | over night at 55-60° C. | | |
| Washing: | 05 min | 2× SSC/0.1% SDS | |
| | Hybridization temperature | | |
| | 30 min | 2× SSC/0.1% SDS | |
| | Hybridization temperature | | |
| | 30 min | 1× SSC/0.1% SDS | |
| | Hybridization temperature | | |
| | 45 min | 0.2× SSC/0.1% SDS | 65° C. |
| | 5 min | 0.1× SSC | room temperature |

The terms “sense” and “antisense” as well as “antisense orientation” are known to the person skilled in the art. Furthermore, the person skilled in the art knows, how long nucleic acid molecules, which are to be used for antisense methods, must be and which homology or complementarity they must have concerning their target sequences.

Accordingly, the person skilled in the art also knows, how long nucleic acid molecules, which are used for other gene silencing methods, must be. For example, the person skilled in the art knows that in the case of an RNAi method, nucleic acid molecules, which are either double stranded RNA one strand of which is homologous or identical, respectively, to an endogenous RNA sequence, or which are DNA molecules, whose transcription in the cell yields corresponding double stranded RNA molecules, must be introduced into the cell, wherein the double stranded RNA molecules inducing the RNA interference usually comprise 20 to 25 nucleotides (see also Tuschl et al., vide supra). A detailed description of this method is also disclosed in WO 99/32619.

A combined application of the above-mentioned methods is also conceivable.

A further object of the invention is a method for increasing the vitamin E content in transgenic plants, wherein, in addition to the alteration of the content and/or the activity of SAT, such enzymes, which cause an increased formation of homogentisate or phytyl pyrophosphate, a reduced degradation of homogentisate or phytyl pyrophosphate or an enhanced conversion within the last steps of the tocopherol biosynthesis (e.g. tocopherol methyltransferase, tocopherol cyclase, γ-tocopherol methyltransferase), are altered regarding their content or their activity in the transgenic plants.

Examples of such enzymes can be found in WO 02/072848, which is in this context hereby explicitly incorporated as disclosure.

Since said enzymes are enzymes which are involved in the regulation of the vitamin E synthesis in vivo, the up-regulation of the content and the activity, respectively, of the above-mentioned enzymes in connection with the alteration of the content and/or the activity of SATs provides further advantages in the production of plants having an increased vitamin E content. In this connection, the up- or down-regulation of the activity and the content, respectively, of said enzymes can be achieved by means of one and/or a combination of the above-mentioned methods.

If, according to the present invention, DNA sequences are used, which are operatively linked in 5′-3′-orientation to a promoter active in plants, vectors can, in general, be constructed, which, after the transfer to plant cells, allow the overexpression of the coding sequence in transgenic plants and plant cells, respectively, or cause the suppression of endogenous nucleic acid sequences, respectively.

Vectors, which can, according to the present invention, be used for overexpression and repression of DNA sequences coding for the different SATs or functionally equivalent parts thereof, can comprise regulatory sequences in addition to the transferred nucleic acid sequences. In this connection, it depends on the aim of the application, which specific regulatory elements and sequences, respectively, are contained in said vectors. Vectors, which can be used for the overexpression of coding sequences in plants, are known to the person skilled in the art. Methods for transferring the sequences as well as for producing transgenic plants and plant cells, respectively, having an increased or decreased expression of proteins, respectively, are also known to the person skilled in the art.

Typically, the regulatory elements contained in vectors ensure the transcription and, if desired, the translation of the nucleic acid sequence, which is transferred to the plants.

These nucleic acid constructs, in which the coding nucleic acid sequences are operatively linked to one or more regulatory signals, which ensure the transcription and the translation in organisms, in particular in plants, are called vectors or also expression cassettes.

Accordingly, the invention further relates to nucleic acid constructs, in particular to nucleic acid constructs functioning as expression cassette, comprising a nucleic acid encoding an SAT or functionally equivalent parts thereof, which is operatively linked to one or more regulatory signals, which ensure the transcription and the translation in organisms, in particular in plants.

Preferably, the regulatory signals contain one or more promoters ensuring the transcription and the translation in organisms, in particular in plants.

The expression cassettes contain regulatory signals, i.e. regulatory nucleic acid sequences, which regulate the expression of the coding sequence in the host cell.

According to a preferred embodiment, an expression cassette comprises a promoter upstream, i.e. at the 5′-end of the coding sequence, and a polyadenylation signal downstream, i.e. at the 3′-end, and optionally comprises further regulatory elements, which are operatively linked to the coding sequence for at least one of the above-mentioned genes located between them.

An operative link is understood to be the sequential arrangement of promoter, coding sequence, terminator and, optionally, further regulatory elements in such a way that each of the regulatory elements can fulfill its function, according to its determination, when expressing the coding sequence.

In a preferred embodiment, the nucleic acid constructs and expression cassettes according to the present invention additionally contain a nucleic acid coding for a peptide, which regulates the localization of the expressed SAT in the cell. Preferably, such nucleic acids code for plastid transit peptides, which ensure the localization in plastids, particularly preferred in chloroplasts, or for signal peptides, which cause the localization in the cytoplasm, the mitochondria or in the endoplasmic reticulum.

In the following, the preferred nucleic acid constructs, expression cassettes, and vectors for plants and methods for producing transgenic plants, as well as the transgenic plants themselves, are described by way of example.

The sequences preferred for operative linking, but not limited thereto, are targeting sequences for ensuring the sub-cellular localization in the apoplast, in the vacuole, in plastids, in the mitochondrion, in the endoplasmic reticulum (ER), in the nucleus, in the oil bodies or other compartments and translation enhancer, like the 5′-leader sequence from the tobacco mosaic virus (Gallie et al., (1987) Nucl. Acids Res. 15, 8693-8711).

Basically, every promoter, which can regulate the expression of foreign genes in plants, is suitable as promoter of the expression cassette. Preferably, a plant promoter or a promoter originating from a plant virus is used in particular. Particularly preferred is the CaMV 35S promoter from the cauliflower mosaic virus (Franck et al., (1980) Cell 21, 285-294). As is known, this promoter contains different recognition sequences for transcriptional effectors, which in their entirety lead to a permanent and constitutive expression of the introduced gene (Benfey et al., (1989) EMBO J. 8 2195-2202). A further possible promoter is the nitrilase promoter.

The expression cassette can also contain a chemically inducible promoter, by which the expression of the target gene in the plant can be regulated at a certain point in time. Such promoters like e.g. the PRP1-promoter (Ward et al., (1993) Plant. Mol. Biol. 22, 361-366), a promoter inducible by salicylic acid (WO 95/19443), a promoter inducible by benzenesulfonamide (EP 388 186), a promoter inducible by tetracycline (Gatz et al., (1992) Plant J. 2, 397-404), a promoter inducible by abscisic acid (EP 335 528) or a promoter inducible by ethanol or cyclohexanone, respectively, (WO 93/21334) can be used.

Furthermore, particularly such promoters are preferred, which ensure the expression in tissues or plant parts, in which e.g. the biosynthesis of vitamin E and its precursors, respectively, takes place. Promoters ensuring a leaf-specific expression are to be mentioned in particular. The promoter of the cytosolic FBPase from potato or the ST-LSI promoter from potato (Stockhaus et al., (1989) EMBO J. 8 2445-245) are to be mentioned.

With the aid of a seed-specific promoter, a foreign protein could be stably expressed to a proportion of 0.67% of the total soluble seed protein in the seeds of transgenic tobacco plants (Fiedler et al., (1995) Bio/Technology 10 1090-1094). Therefore, the expression of SATs in the seed of plants using seed-specific promoters, like e.g. the phaseolin (U.S. Pat. No. 5,504,200), the USP (Baumlein et al., (1991) Mol. Gen. Genet. 225 (3), 459-467), the LEB4, the vicilin and the legumin B4 promoter, is particularly preferred.

Biosynthesis of vitamin E in plants occurs, inter alia, in the leaf tissue, so that a leaf-specific expression of the nucleic acids according to the present invention, which encode an SAT, is useful. However, this is not restrictive, since the expression can also occur in every other part of the plant—in particular in fatty seeds—in a tissue-specific manner.

Therefore, a further preferred embodiment relates to a seed-specific expression of the above-described nucleic acids.

Furthermore, a constitutive expression of the SAT is advantageous. On the other hand, an inducible expression may also seem desirable.

The efficiency of the expression of the transgenically expressed SAT can e.g. also be determined in vitro by means of shoot meristem propagation. In addition, an expression, altered in manner and in amount, of the SAT and its effect on the capacity of vitamin E biosynthesis can be tested with test plants in greenhouse experiments.

The promoter can be both native and homologous, respectively, and foreign and heterologous, respectively, in relation to the host plant. In the 5′-3′-transcription direction, the expression cassette preferably contains the promoter, a coding nucleic acid sequence, and possibly a region for the transcriptional termination. Different termination regions are exchangeable by one another as desired.

Preferred polyadenylation signals are plant polyadenylation signals, preferably such, which substantially correspond to T-DNA-polyadenylation signals from Agrobacterium tumefaciens, particularly of the gene 3 of the T-DNA (Octopin Synthase) of the Ti-plasmid pTiACH5 (Gielen et al., (1984) EMBO J. 3, 835 ff.) or functional equivalents thereof.

The production of an expression cassette is preferably carried out by fusion of a suitable promoter with an above-described nucleic acid like a sequence coding for SAT and preferably a target nucleic acid inserted between promoter and nucleic acid sequence, which e.g. codes for a chloroplast-specific transit peptide, and a polyadenylation signal, in accordance with common recombination and cloning techniques, as e.g. described in Sambrook et al., (vide supra) and in T. J. Silhavy; M. L. Berman and L. W. Enquist, Experiments with Gene Fusions, Cold Spring Harbor Laboratory, Cold Spring Harbor, N.Y. (1984) and in Ausubel et al., Current Protocols in Molecular Biology, Greene Publishing Assoc. and Wiley-Interscience (1987).

Particularly preferred are inserted target nucleic acids, which ensure a targeting into the plastids.

It is also possible to use expression cassettes, whose nucleic acid sequence codes for a fusion protein, wherein a part of the fusion protein is a transit peptide regulating the translocation of the polypeptide. Preferred are transit peptides, which are specific for the chloroplasts and which are enzymatically cleaved from the target protein part after the translocation of the target protein into the chloroplasts.

Likewise preferred, the expression cassettes contain sequences coding for a fusion protein with a cytoplasm peptide. In this connection, the localization in the cytoplasm possibly can also be ensured by leaving out the sequence for the plastid transit peptide.

Particularly preferred is the transit peptide derived from the plastid Nicotiana tabacum transketolase or from another transit peptide (e.g. the transit peptide of the small subunit of rubisco (rbcs) or of the ferredoxine NADP oxidoreductase as well as of the isopentenyl pyrophosphate isomerase-2) or from a functional equivalent thereof.

The nucleic acids according to the present invention can be produced synthetically or obtained naturally or they can contain a mixture of synthetic and natural nucleic acid components and they can consist of different heterologous gene sections of different organisms.

Preferred are, as described above, synthetic nucleotide sequences with codons, which are preferred by plants. These codons preferred by plants can be determined from codons having the highest protein frequency, which are expressed in most of the plant species of interest.

When preparing an expression cassette, different DNA fragments can be manipulated in order to obtain a nucleotide sequence, which advisably reads in the correct direction and which is equipped with a correct reading frame. Adaptors or linkers can be attached at the fragments for connecting the DNA fragments with each other.

Advisably, the promoter and terminator regions in the direction of transcription can be equipped with a linker or a polylinker, which contains one or more restriction sites for the insertion of said sequence. Normally, the linker has 1 to 10, mostly 1 to 8, preferably 2 to 6 restriction sites. Within the regulatory regions, the linker generally has a size of less than 100 bp, often less than 60 bp, at least, however, 5 bp.

Furthermore, manipulations providing suitable restriction sites or removing superfluous DNA or restriction sites can be utilized. Where insertions, deletions, or substitutions like e.g. transitions or transversions are possible, in vitro mutagenesis, primer repair, restriction, or ligation can be used.

In the case of suitable manipulations, like e.g. restriction, chewing-back, or filling in of overhangs for blunt ends, complementary ends of the fragments can be provided for the ligation.

Therefore, the invention relates to vectors comprising the above-described nucleic acids, nucleic acid constructs, or expression cassettes.

The transfer of foreign genes into the genome of an organism, in particular a plant, is referred to as transformation.

To this end, methods known per se for transforming and regenerating plants from plant tissues or plant cells can be used for the transient or stable transformation, in particular with plants.

Suitable methods for transforming plants are the protoplast transformation by polyethylene glycol-induced DNA uptake, the biolistic method with the gene gun—the so-called particle bombardment method, the electroporation, the incubation of dry embryos in DNA-containing solution, the microinjection and the gene transfer mediated by Agrobacterium. The mentioned methods are e.g. described in B. Jenes et al., (1993) Techniques for Gene Transfer, in: Transgenic Plants, Vol. 1, Engineering and Utilization, published by S. D. Kung and R. Wu, Academic Press, 128-143 and in Potrykus et al., (1991) Annu. Rev. Plant Physiol. Plant Molec. Biol. 42, 205-225).

In connection with the injection and electroporation of DNA in plant cells, no specific requirements are actually made for the used plasmids. Similarly, this applies to the direct gene transfer. Simple plasmids like e.g. pUC derivatives can be used. Typically, vectors can be used, which have sequences required for the propagation and selection in E. coli. Belonging thereto are also vectors of the pBR322, M13m series and pACYC 184. However, if entire plants are to be regenerated from cells transformed in such a way, the presence of a selectable marker gene is required. The commonly used selection markers are known to the person skilled in the art and selecting a suitable marker does not pose a problem. Common selection markers are such, which confer resistance against a biocide or an antibiotic like kanamycin, G418, bleomycin, hygromycin, methotrexate, glyphosate, streptomycin, sulfonyl urea, gentamycin or phosphinotricin and the like to the transformed plant cells.

Depending on the method of introduction of desired genes into the plant cell, further DNA sequences may be required. If, for example, the Ti or the Ri plasmid are used for the transformation of the plant cell, at least the right side border, though often the right and left side borders, of the T-DNA included in the Ti and Ri plasmid, have to be joined with the genes that are to be introduced to form a flanking region.

If agrobacteria are used for the transformation, the DNA that is to be introduced has to be cloned into specific plasmids, actually either into an intermediate or into a binary vector. Due to sequences, which are homologous to sequences in the DNA, the intermediate vectors can be integrated into the Ti or Ri plasmid of the agrobacteria by means of homologous recombination. Said plasmid also contains the vir region necessary for the transfer of the T-DNA. Intermediate vectors cannot replicate in agrobacteria. By means of a helper plasmid, the intermediate vector can be transferred to Agrobacterium tumefaciens (conjugation).

Binary vectors can replicate in both E. coli and in agrobacteria. They contain a selection marker gene and a linker or polylinker, which are framed by the left and right T-DNA border region. They can be transformed directly into the agrobacteria (Holsters et al., (1978) Molecular and General Genetics 163, 181-187). The agrobacterium serving as a host cell should contain a plasmid carrying a vir region. The vir region is necessary for the transfer of the T-DNA into the plant cell. T-DNA can additionally be present. The agrobacterium transformed in such a way is used for the transformation of plant cells.

The use of T-DNA for the transformation of plant cell has been intensely examined and sufficiently described in EP 120 515.

For the transfer of the DNA into the plant cell, plant explants can advisably be cultivated with Agrobacterium tumefaciens or Agrobacterium rhizogenes. The transformation of plants by agrobacteria is, inter alia, known from F. F. White, Vectors for Gene Transfer in Higher Plants; in Transgenic Plants, Vol. 1, Engineering and Utilization, published by S. D. Kung and R. Wu, Academic Press, 1993, S. 15-38.

Entire plants can then be regenerated from the infected plant material (e.g. pieces of leaves, segments of stems, roots, but also protoplasts or suspension-cultivated plant cells) in a suitable medium, which can contain antibiotics or biocides for the selection of transformed cells. The regeneration of the plants is carried out according to common regeneration methods using known nutritional media. The plants and plant cells, respectively, thus obtained can then be examined concerning the presence of the introduced DNA.

Using the above-cited recombination and cloning techniques, the expression cassettes can also be cloned into suitable vectors, which allow their propagation, e.g. in E. coli. Suitable cloning vectors are, inter alia, pBR332, pUC series, M13mp series and pACYC 184. Particularly suitable are binary vectors, which can replicate in E. coli as well as in agrobacteria.

By way of example, the plant expression cassette can be incorporated into a derivative of the transformation vector pSUN2 having a vicilin promoter (WO 02/00900).

While the transformation of dicotyledonous plants and their cells, respectively, via Ti plasmid vector systems with the aid of Agrobacterium tumefaciens is well established, recent studies indicate that also monocotyledonous plants and their cells, respectively, are indeed accessible for the transformation by means of vectors based on agrobacteria (see inter alia Chan et al., (1993), Plant Mol. Biol. 22, 491-506).

Alternative systems for the transformation of monocotyledonous plants and cells thereof, respectively, are the transformation by means of the biolistic approach (Wan et al., (1994) Plant Physiol. 104, 37-48; Vasil et al., (1993) Bio/Technology 11, 1553-1558; Ritala et al., (1994) Plant Mol. Biol. 24, 317-325; Spencer et al., (1990) Theor. Appl. Genet. 79, 625-631), the protoplast transformation, the electroporation of partially permeabilized cells and the introduction of DNA by means of glass fibers (vgl. L. Willmitzer (1993) Transgenic Plants in: Biotechnology, A Multi-Volume

Comprehensive Treatise (Publisher: H. J. Rehm et al., Band 2, 627-659, VCH Weinheim, Germany).

The transformed cells grow inside the plant in the usual manner (see also McCormick et al., (1986) Plant Cell Reports 5, 81-84). The resulting plants can be raised normally and can be crossed with plants having the same transformed hereditary factor or other hereditary factors. The hybrid individuals resulting therefrom have the corresponding phenotypic features.

Two or more generations should be raised in order to ensure that the phenotypic feature is stably maintained and inherited. Seeds should also be harvested in order to ensure that the corresponding phenotype or other features have been maintained.

According to usual methods, transgenic lines can also be determined, which are homozygous for the new nucleic acid molecules, and their phenotypic behavior concerning an increased vitamin E content can be examined and compared to the behavior of hemizygous lines.

Of course, plants containing the nucleic acid molecules according to the present invention can also be continued to be cultivated as plant cells (including protoplasts, calli, suspension cultures and the like).

The expression cassette can also be utilized beyond the plants for the transformation of bacteria, in particular cyanobacteria, mosses, yeasts, filamentous fungi, and algae.

Therefore, the invention further relates to the use of the above-described nucleic acids and the above-described nucleic acid constructs, in particular of the expression cassettes for producing genetically engineered organisms, in particular for producing genetically engineered plants or for transforming plant cells, plant tissues or plant parts.

Preferably, said transgenic plants have an increased vitamin E content in comparison with the wild type.

Therefore, the invention further relates to the use of SATs or of the nucleic acid constructs according to the present invention for increasing the vitamin E content in organisms, which are capable of producing vitamin E as wild type.

It is known that plants with a high vitamin E content exhibit an increased resistance against abiotic stress. Abiotic stress is understood to mean e.g. cold, frost, drought, heat and salt.

Therefore, the invention further relates to the use of the nucleic acids according to the present invention for producing transgenic plants, which have an increased resistance against abiotic stress in comparison with the wild type. The above-described proteins and nucleic acids can be used for producing fine chemicals in transgenic organisms, preferably for producing vitamin E in transgenic plants.

Preferably, the goal of the use is to increase of the vitamin E content of the plant or the plant parts.

Depending on which promoter is chosen, the gene can be expressed specifically in the leaves, in the seeds, petals or in other parts of the plant.

Accordingly, the invention further relates to a method for producing genetically engineered organisms by means of introducing an above-described nucleic acid or an above-described nucleic acid construct or an above-described combination of nucleic acid constructs into the genome of the original organism.

The invention further relates to the above-described genetically engineered organisms themselves.

As mentioned above, the genetically engineered organisms, in particular plants, have an increased vitamin E content.

In a preferred embodiment, as mentioned above, photosynthetically active organisms like e.g. cyanobacteria, mosses, algae or plants, particularly preferred plants, are used as original organisms for producing organisms having an increased vitamin E content in comparison with the wild type.

The plants used for the method according to the present invention can, in principle, be any desired plant. Preferably, it is a monocotyledonous or dicotyledonous crop plant, food plant or forage plant. Examples for monocotyledonous plants are plants belonging to the genera of avena (oats), triticum (wheat), secale (rye), hordeum (barley), oryza (rice), panicum, pennisetum, setaria, sorghum (millet), zea (maize) and the like.

Dicotyledonous crop plants comprise inter alia cotton, leguminoses like pulse and in particular alfalfa, soy bean, rapeseed, tomato, sugar beet, potato, ornamental plants as well as trees. Further crop plants can comprise fruits (in particular apples, pears, cherries, grapes, citrus, pineapple and bananas), oil palms, tea bushes, cacao trees and coffee trees, tobacco, sisal as well as, concerning medicinal plants, rauwolfia and digitalis. Particularly preferred are the grains wheat, rye, oats, barley, rice, maize and millet, sugar beet, rapeseed, soy, tomato, potato and tobacco. Further crop plants can be taken from U.S. patent U.S. Pat. No. 6,137,030.

Preferred plants are tagetes, sunflower, arabidopsis, tobacco, red pepper, soy, tomato, eggplant, pepper, carrot, small carrot, potato, maize, lettuces and types of cabbage, grains, alfalfa, oats, barley, rye, wheat, triticale, millet, rice, lucerne, flax, cotton, hemp, brassicacea like e.g. rapeseed or canola, sugar beet, sugar cane, species of nuts or wine or wood plants like e.g. aspen or yew.

Particularly preferred are Arabidopsis thaliana, Tagetes erecta, Brassica napus, Nicotiana tabacum, sunflower, canola, potato or soy.

Such transgenic plants, their propagation material and their plant cells, plant tissues or plant parts are further objects of the present invention.

Therefore, the invention also relates to harvest products and propagation material of transgenic plants, which have been produced according to a method according to the present invention and which have an increased vitamin E content. The harvest products and the propagation material are in particular fruits, seeds, blossoms, tubers, rhizomes, seedlings, cuttings, etc. Parts of said plants, like plant cells, protoplasts and calli can also be used.

The genetically engineered organisms, in particular plants, can be used for producing vitamin E, as is described above.

Genetically engineered plants according to the present invention, which have an increased vitamin E content and can be consumed by humans and animals, can e.g. also be used directly or after processing known per se as food or feed or as feed and food additive.

The genetically engineered plants according to the present invention can further be used for producing vitamin E-containing extracts.

Within the scope of the present invention, increase of the vitamin E content preferably means the artificially acquired capability of an increased biosynthesis capacity of said compounds in the plant in comparison with the plant not modified by genetic engineering, preferably for the duration of at least one plant generation.

Normally, an increased vitamin E content is understood to be an increased content of total tocopherol. In particular, an increased vitamin E content is also understood to be an altered content of the above-described eight compounds having tocopherol activity.

The determination of the vitamin E content is carried out according to methods common in the art. These are, in particular, disclosed in detail in WO 02/072848, whose content is hereby explicitly referred to as disclosure of methods for detecting the vitamin E content in plants.

The vitamin E content can be determined e.g. in leaves and seeds of plants transgenic for SATs. To this end, in particular dry seeds or frozen leaf material is used.

The leaf material of the plants is deep-frozen in liquid nitrogen immediately after taking the sample. The subsequent breaking of the cells (leaves or seeds) is carried out by means of a stirring device by triple incubation in the Eppendorf shaker at 30° C., 1000 rpm (revolutions per minute) in 100% methanol for 15 minutes, wherein the respectively obtained supernatants are combined. Normally, further incubation and extraction steps do not yield further release of tocopherols or tocotrienols.

In order to avoid oxidation, the obtained extracts are analyzed immediately after the extraction by means of an HPLC device (Waters Allience 2690). Tocopherols and tocotrienols are separated via a common reverse phase column (ProntoSil 200-3-C30 TM, by Bischoff) with a mobile phase of 100% methanol and identified by means of standards (by Merck). The fluorescence of the substances (excitation 295 nm, emission 320 nm), which can be detected by means of a Jasco fluorescence detector FP 920, serves as detection system.

The present invention is explained in the following examples, which only serve the purpose of illustrating the invention and are by no ways to be understood as limitation.

### EXAMPLES

General Cloning Methods:

Cloning methods like e.g. restriction cleavage, DNA isolation, agarose gel electrophoresis, purification of DNA fragments, transfer of nucleic acids to nitrocellulose and nylon membranes, linking of DNA fragments, transformation of E. coli cells, raising of bacteria, sequence analysis of recombinant DNA, were carried out according to Sambrook et al., vide supra. The transformation of Agrobacterium tumefaciens was carried out according to the method described by Höfgen et al., ((1988) Nucl. Acids Res. 16, 9877). The raising of agrobacteria was carried out in YEB medium (Vervliet et al., (1975) J. Gen. Virol. 26, 33).

Bacteria Strains and Plasmids

E. coli (XL 1 Blue) bacteria were obtained from Stratagene, La Jolla, USA. The agrobacteria strain used for plant transformation (GV3101; Bade and Damm in Gene Transfer to Plants; Protrykus, I. and Spangenberg, G., eds., Springer Lab Manual, Springer Verlag, 1995, 30-38) was transformed with the vector pSUN2. The vector pSUN2 was used for cloning.

Production of Transgenic Rapeseed Plants (Brassica Napus)

Transgenic oilseed rapeseed plants were produced according to a standard protocol (Bade and Damm in Gene Transfer to Plants; Protrykus, I. and Spangenberg, G., eds., Springer Lab Manual, Springer Verlag, 1995, 30-38). Said reference also discloses the composition of the used media and buffers.

The seeds of Brassica napus var. Westar were surface-sterilized with 70% ethanol (v/v), washed in water at 55° C. for 10 min and incubated in a 1% hypochlorite solution (25% (v/v) Teepol, 0.1% (v/v) Tween 20) for 20 min. Subsequently, each seed was washed six times with sterile water for 20 min. The seeds were dried on filter paper for three days. 10 to 15 seeds were then germinated in a glass vessel containing 15 ml germination medium. The roots and apices were removed from different seedlings (size about 10 cm) and the remaining hypocotyls were cut into small pieces of about 6 mm in length. The 600 explants thus obtained were washed in 50 ml basal medium for 30 min and then transferred into a 300 ml container. After adding 100 ml callus induction medium, the cultures were incubated while being shaken at 100 rpm (revolutions per minute) for 24 h.

For transformation, an overnight culture of Agrobacterium tumefaciens was raised in Luria Broth medium, which contained kanamycin (20 mg/l), at 29° C. and 2 ml of this culture were incubated in 50 ml antibiotics-free Luria Broth medium at 29° C. for 4 h, until an OD₆₀₀ of 0.4 to 0.5 was reached. The culture was then centrifuged at 2000 rpm for 25 min and the cell pellet was resuspended in 25 ml Basal medium. The concentration of the bacteria in the solution was adjusted to an OD₆₀₀ of 0.3 by corresponding addition of medium.

The callus induction medium was removed from the oilseed rapeseed explants by means of sterile pipettes and 50 ml of the bacteria suspension were added to the explants. The reaction mixture was then mixed carefully and incubated for 20 min. The bacteria suspension was then removed and the oilseed rapeseed explants were subsequently washed with 50 ml of the callus induction medium for 1 min. Subsequently, 100 ml of the callus induction medium were added. The co-cultivation was carried out while shaking at 100 rpm for 24 h and stopped by removal of the callus induction medium. The explants were then each washed twice with 25 ml washing medium for 1 min and twice with 100 ml washing medium for 60 min while being shaken at 100 rpm. Together with the explants, the washing medium was then transferred to 15 cm petri dishes and the medium was removed by means of sterile pipettes.

For regeneration, 20 to 30 explants in each case were transferred to 90 mm petri dishes containing 25 ml shoot induction medium with kanamycin. The petri dishes were sealed with two layers of Leukopor® and subjected to a photocycle of 16 h light and 16 h darkness at 25° C. and 2000 Lux. In each case, the developing calli were transferred to fresh petri dishes also containing shoot induction medium after 12 days. All further steps for the regeneration of complete plants were carried out as described in the above-mentioned reference (Bade and Damm, vide supra).

Production of an Enzymatically Inactive Serine Acetyltransferase (SAT), Which Still is Capable of Interacting with OAS-TL

The SAT-A from Arabidopsis thaliana, which is described in the art regarding its amino acid sequence and the underlying DNA sequence, (EMBL Accession code X82888; Bogdanova and Hell (1995) Plant Physiol. 109, 1498; Wirtz et al., 2002, vide supra) was inactivated by means of directed mutagenesis of the amino acid histidine 309 to alanine (the numbering refers to the first methionine of the open reading frame). The directed mutagenesis of the corresponding cDNA in the plasmid pBlueScript (Stratagene) was carried out by means of base pair substitution according to a commercially available method of Promega (Heidelberg, Germany).

The site-directed mutagenesis of the SAT-A cDNA was carried out with pBS/ΔSAT1-6 (Bogdanova et al., (1995) FEBS Lett. 358, 43-47). The employed Promega GeneEditor in vitro Site Directed Mutagenesis System achieved an average of 80% positive clones. The point mutations were verified by means of DNA sequencing and the resulting amino acid substitutions were numbered with reference to the start codon of the longest possible open reading frame of a mitochondrial SAT-A cDNA (cDNA SAT-1 (Roberts et al., (1996) Plant Mol. Biol. 30, 1041-1049)).

The inactivation of the SAT mutant by means of the amino acid substitution at position 309 (histidine→alanine) was confirmed by absent heterologous complementation of an SAT-free E. coli mutant and by enzyme determination in vitro (maximum of 1% residual activity). The capability of interacting with O-acetylserine (Thiol) lyase (OAL-TL) was proven by heterologous expression in the yeast “two-hybrid” system and by co-expression in E. coli with subsequent biochemical purification.

The inactivation of the cysE gene from E. coli in order to produce an SAT-free E. coli mutant was carried out according to the method described by Hamilton et al. (Hamilton et al. (1989) J. Bacteriol. 171, 4617-4622). Hereby, a bacteria strain was to be provided, which is, regarding its SAT deficiency, more stable than those presently available in the art. In order to achieve this, the wild type cysE gene was cloned by means of PCR and inactivated by means of insertion of a gentamycin resistance cassette into a Clal restriction site at position 522, relative to the starting codon of the cysE gene.

After cloning this cassette into the plasmid pMAK705, which has a replication origin that is sensitive to temperature, the inactivated cysE gene was integrated into the genome of E. coli C600 via homologous recombination in order to form the strain MW1 (thr, leu, thi, lac, λ-P1+F′, cysE, Gm^{r}). Complementation tests using the E. coli strains EC1801 (E. coli Genetic Stock Center, Yale University, New Haven, Conn., USA) or MW1 were carried out on M9 minimal medium agar plates with or without cysteine while adding induction agent and selective antibiotics.

The constructs for the expression of the mitochondrial SAT-A from Arabidopsis thaliana comprised pBS/ΔSAT1-6 (X82888; Bogdanova et al., (1985) vide supra), pET/ΔSAT1-6 and mutated forms of the SAT-A. In order to obtain the latter plasmid, the coding region of the SAT-A was amplified by means of PCR from base pair 28-939 without mitochondrial transit peptide using specific primers, flanked by EcoRI and XhoI sites. This fragment was cloned into the plasmid pCAP (Roche, Mannheim, Germany), the sequence was verified by means of sequencing of both strands and inserted into the corresponding sites of pET29a (Novagen, Madison, USA), which resulted in a fusion protein having the 35 amino acids of the S-tag at its N-terminus for affinity purification on S-agarose. Mitochondrial OAS-TL from A. thaliana was expressed in a similar manner by means of cloning of a PCR product, which comprised the mature protein without the mitochondrial transit peptide from base pair 172-1162 (AJ271727 (Hesse et al., (1999) Amino Acids 16, 113-131), into the NcoI-BamHI sites of pET3d, which resulted in pET/OAS-C.

Expression, cultivation and affinity purification of SAT and OAS-TL using the S-tag system (Novagen) were essentially carried out as described by Droux et al., (1998, Eur. J. Biochem. 255, 235-245), with the following modifications. After the last washing step, the S-tag was not removed by means of proteolytic cleavage at the affinity column, as this treatment resulted in fractions with labile SAT activity. Instead, SAT was eluted with 3 M MgCl₂, which was subsequently removed by means of gel filtration at PD10 columns (Amersham, Freiburg, Germany). In vitro interaction of SAT and OAS-TL at the column was determined according to a standard washing and elution protocol with or without 1 mM OAS (O-acetylserine), as described by Droux et al., (1998, vide supra).

The determination of protein concentration and the separation of proteins were carried out according to standard protocols (e.g. Sambrook et al., vide supra).

The SAT enzymatic activity with and without OAS-TL was determined in a standard assay on the basis of the method according to Kredich and Becker (1971, In Methods in Enzymology (Tabor and Tabor, eds), pages 459-469, Academic Press, New York, USA). Raw or purified recombinant SAT protein was incubated in a volume of 250 μl (50 mM tris/HCl, pH 7.5, 0.2 mM acetyl-CoA, 2 mM dithiothreitol, 5 mM serine) at 25° C. and A₂₃₂ was recorded for up to 3 min.

The OAS-TL activity was examined under saturated conditions, as described before (Nakamura et al., (1987) Plant Cell Physiol. 28, 885-891). Kinetic analyses were carried out with the SigmaPlot software, which allowed hyperbolic adaptations on the basis of the Michaelis-Menten equation:
v=Vₘₐₓ×([S]/(Kₘ+[S]).
For the interaction analyses using the yeast two-hybrid system, the transformations of the yeast strains HF7c and PCY2, the selection on minimal medium and β-galactosidase assays were carried out as already described (Bogdanova and Hell (1997) Plant J. 11, 251-262). PCR with specific primer pairs flanked by Sall and Spel sites, respectively, were used for all of the constructs in order to insert the coding regions into the corresponding restriction sites of pPC86 (GAL4 activation domain) and pPC97 (GAL4-DNA binding domain) (Chevray and Nathans (1992) Proc. Natl. Acad. Sci. USA 89, 5789-5793). EST 181H17T7 (GenBank Accession Number AJ2711727) was used as template in order to generate OAS-TL C without mitochondrial transit peptide from base pair 172-1162. In contrast to the hitherto used full length construct (Bogdanova et al., (1997) vide supra), the pPC vectors with mitochondrial SAT-A without mitochondrial transit peptide were constructed by amplification of base pair 28-939 (X82888 (Bogdanova et al., (1995) vide supra).
Expression of the Active SAT-A and of the Non-Functional SAT-A Mutant (H309A) in Plants

The cDNAs of the active SAT-A (SAT) and the inactivated mutant SAT-A H309A (SATH309A) were cloned into a binary transformation vector (pSUN2, WO 02/00900). SAT and SATH309A were amplified by PCR in the same manner and fused with the reading frame of the rbcs transit peptide. The localization of both SATs in the cytosol was carried out using pSUN2 while omitting the region for the import peptide. Either the nitrilase promoter for a constitutive expression or the vicilin promoter for a seed-specific expression were used as promoters.

In each case, the clonings were carried out into the pre-determined Xhol and Smal restriction sites, respectively, of said vector pSUN2.

In each case, the cDNA of the active SAT and the SAT mutant SATH309A was amplified with the oligonucleotide primers SAT269 and SAT270, which had 5′-located additional XhoI and EcoRI restriction sites, by means of standard PCR. After digestion with EcoRI, the SAT fragments were fused with the likewise EcoRI-digested transit peptide rbcs by ligation. The transit peptide was likewise amplified by means of the oligonucleotide primers Tra201 and Tra202, which had 5′-located additional EcoRI and SmaI restriction sites, by means of standard PCR. Subsequently, the ligation of the fused fragments into the Xhol and Smal restriction sites of the vector was carried out.

Example for standard PCR: Reaction volume 50 μl with 20 pmol of each primer, 1-10 ng plasmid, buffer of the manufacturer, 1 U Taq polymerase (Promega). Sequence: 5 min at 94° C., then 30 cycles of 30 sec at 94° C., 60 sec at 55° C., 30 sec at 72°, followed by 10 min at 72° C.

Tra201: 5′-CTC GAG AAT GGC TTC CTC AAT G-3′
Tra202: 5′-GAA TTC CCA CAC CTG CAT GCA TTG
TAC TC-3′
SAT269: 5′-GAA TTC CAT GAA CTA CTT CCG TTA
TC-3′
SAT270: 5′-CCC GGG TCA AAT TAC ATA ATC CGA
C-3′

Extraction and Determination of Activity of the SATs from Transgenic Rapeseed Plants

The SATs were extracted from the transgenic plant material and their activity was determined. To this end, the protocol by Nakamura et al., ((1987) Plant Cell Physiol., 28, 885-891) was used. In each case, the leaves (nitrilase promoter) or the seeds (vicilin promoter) of three independent transgenic lines were examined. It turned out that transgenic plants expressing the SATH309A have an unaltered SAT activity in comparison with non-transgenic plants, while transgenic plants overexpressing the active SAT have a significantly increased activity of total-SAT.

Determination of the Vitamin E Content of the Transgenic Plants

The extraction of vitamin E and its detection was carried out as described above. Frozen leaf material (nitrilase promoter) or dry seeds (vicilin promoter) were used for the analysis. The leaf material of the plants was correspondingly deep-frozen in liquid nitrogen immediately after taking the sample. The subsequent breaking of the cells (leaves or seeds) was carried out by means of a stirring device by triple incubation in the Eppendorf shaker at 30° C., 1000 rpm (revolutions per minute) in 100% methanol for 15 minutes, wherein the respectively obtained supernatants were combined. Normally, further incubation and extraction steps did not yield further release of tocopherols or tocotrienols.

In order to avoid oxidation, the obtained extracts were analyzed immediately after extraction by means of an HPLC device (Waters Alliance 2690). Tocopherols and tocotrienols were separated via a common reverse phase column (ProntoSil 200-3-C30 TM, by Bischoff) with a mobile phase of 100% methanol and identified by means of standards (by Merck). The fluorescence of the substances (excitation 295 nm, emission 320 nm), which was detected by means of a Jasco fluorescence detector FP 920, served as detection system.

An increase of the vitamin E content in comparison with the wild type could be detected in both the transgenic plant material with active SAT or inactive SATH304A, regardless of whether a constitutive or a seed-specific expression had been carried out.

The above-described results clearly showed that the Arabidopsis thaliana SAT mutant having an amino acid substitution at position 309 has no enzymatic activity anymore, but is still capable of forming complexes, i.e. of interacting with OAS-TL, though. The expression of this mutant, just like the expression of an active SAT, led to a surprising increase of the vitamin E content.

### FIGURES

FIG. 1 shows typical pathways of vitamin E biosynthesis.

FIG. 2 shows an amino acid alignment of different serine acetyltransferases.

FIG. 3 shows a vector map of the pSUN2 with the rbcs-SATH309A construct.

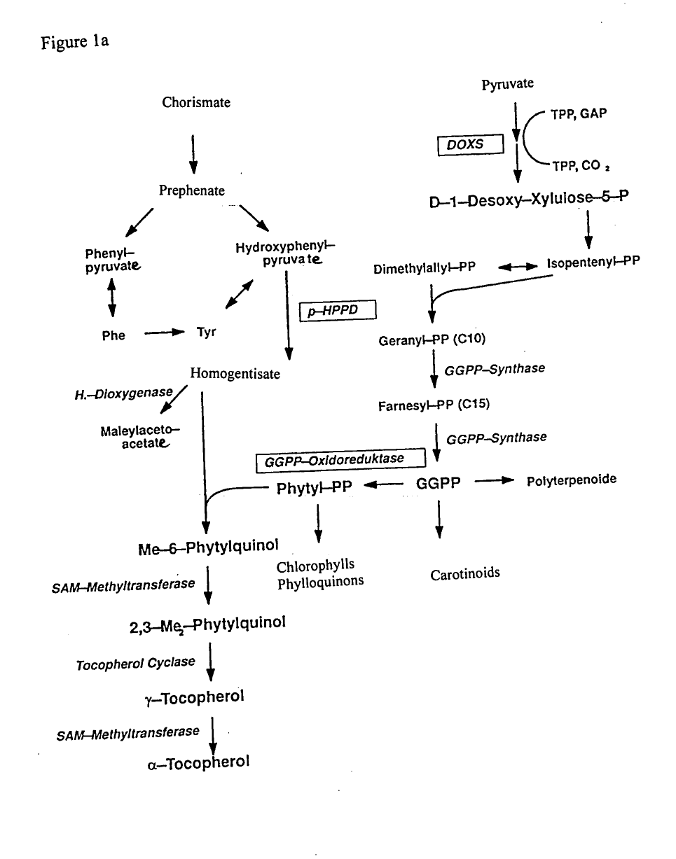

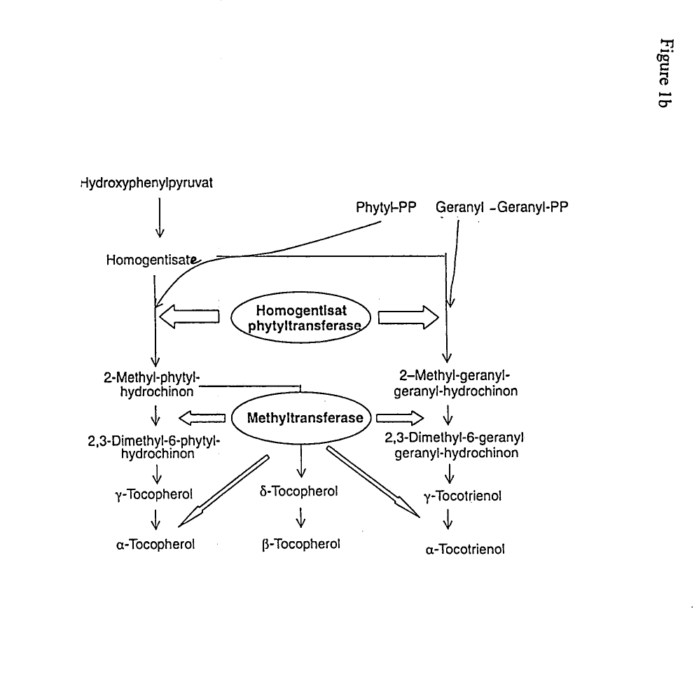

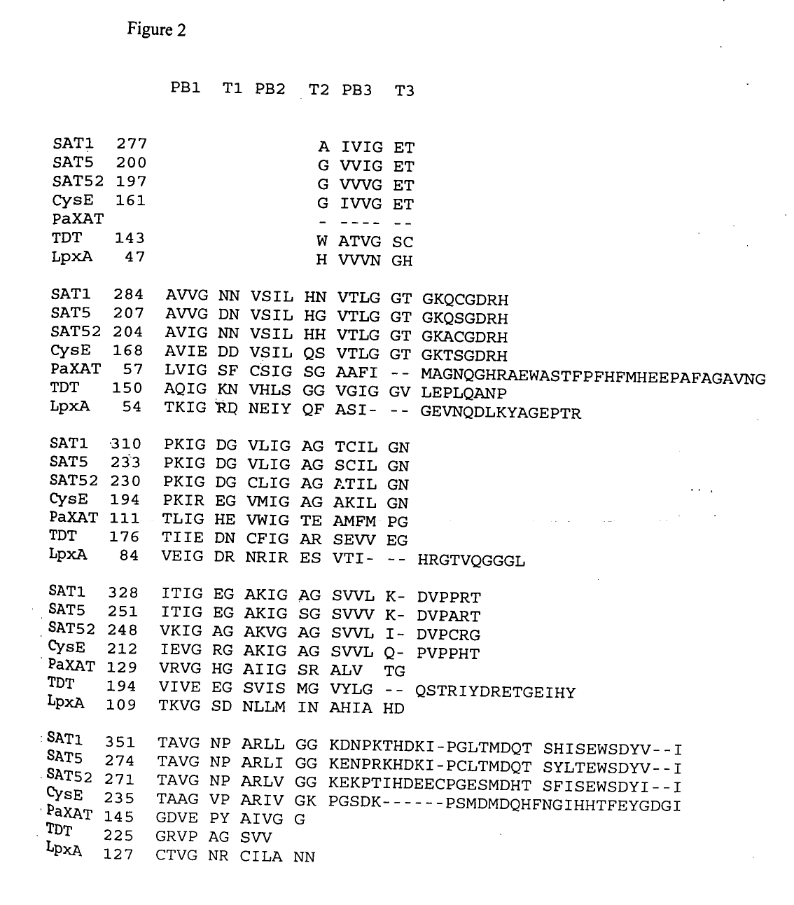

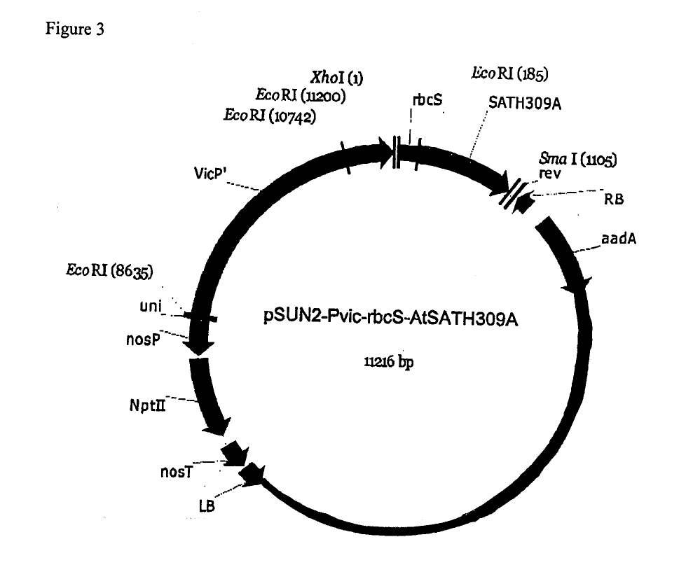

1. A method for increasing the vitamin E content in transgenic plants and/or plant cells, comprising altering the content and/or the activity of serin acetyl transferase (SAT) in the transgenic plants and/or plant cells in comparison to the wild-type.

2. The method according to claim 1, wherein the SAT content is increased by transferring a nucleic acid encoding an SAT or a functionally equivalent part thereof to the plant or to the plant cell.

3. The method according to claim 1, wherein the SAT is a feedback-regulated and/or a feedback-independent SAT.

4. The method according to claim 1, wherein the SAT is an SAT from microorganisms, from fungi, or from plants, or hybrids.

5. The method according to claim 4, characterized in that the SATs are the SATs with the Genbank accession numbers (in brackets: gene annotations of the Arabidopsis genome sequencing) L42212(At1g55920), AF112303(At2g17640), X82888(At3g13110), U30298(At5g56760), At4g35640, AJ414051, AJ414052, AJ414053 or SATs with sequences which are substantially homologous to the sequences with the mentioned accession numbers.

6. The method according to claim 1, wherein the SATs are non-functional SATs having point mutation(s), deletions and/or insertions.

7. The method according to claim 6, wherein the SAT is a non-functional SAT, which is enzymatically inactive due to a mutation within the amino acid sequence motif of SEQ ID NO: 1.

8. The method according to claim 7, wherein the mutation is within the core motif of SEQ ID NO: 2.

9. The method according to claim 7, wherein the histidine within the motif is mutated.

10. The method according to claim 1, comprising the following steps:
a) Production of a vector comprising the following nucleic acid sequences in 5′-3′ orientation:
a promoter sequence functional in plants
operatively linked thereto a DNA sequence encoding an SAT or functionally equivalent parts thereof
a termination sequence functional in plants
b) Transfer of the vector from step a) to a plant cell.

11. The method according to claim 10, wherein the vector additionally has nucleic acid sequences which effect the compartment-specific expression of the SAT in the transgenic plant and/or plant cell.

12. The method according to claim 1, wherein the content and/or the activity of the endogenous SATs is altered in comparison to the wild-type.

13. The method according to claim 12, wherein the content and/or the activity of the endogenous SATs is increased by influencing the transcription and/or translation.

14. The method according to claim 12, wherein the content and/or activity of the endogenous SATs is increased by regulation of the post-translational modifications.

15. The method according to claim 1, wherein the transgenic plants and/or plant cells are harvested after cultivation and wherein vitamin E is subsequently isolated from the plants and/or plant cells.

16. The method according to claim 1, wherein the plants are monocotyledonous or dicotyledonous plants.

17. The method according to claim 16, wherein the transgenic plants are cotton, leguminous plants, soy, rapeseed, tomato, sugarbeet, potato, tobacco, sisal or grains.

18. The method according to claim 1, wherein the content of thiol compounds is altered within the plants and/or plant cells compared to the wild-type.

19. The method according to claim 18, wherein the content of glutathione, S-adenosylmethionine, methionine and cysteine is altered within the plants and/or plant cells compared to the wild-type.

20. (canceled)

21. (canceled)

22. (canceled)

23. The method, according to claim 4, wherein the SAT is an SAT selected from E. coli, Corynebacterium glutamicum, Saccharomyces cerevisiae, Schizosaccharomyces pombe, Aspergillus nidulans, Neurospora crassa, Arabidopsis thaliana, Nicotiana tabacum, Allium tuberosum, Brassica oleracea, Glycine max, Zea mays, and Triticum aestivum.

24. The method according to claim 10, further comprising the integration of the transferred vector into the plant genome.

25. The method according to claim 11, wherein the vector has nucleic acid sequences that effect the compartment-specific expression of the SAT in mitochondria, plastids, chloroplasts and/or the cytosol.

26. The method according to claim 17, wherein the transgenic plants are wheat, rye, oats, barley, rice, maize, or millet.


SEQUENCE LISTING (6 sequences)

SEQ ID NO: 1 - protein, 13 aa, Plant
  PEPTIDE (1)..(13): Amino acid sequence motif
GKXXGDRHPKIGD

SEQ ID NO: 2 - protein, 3 aa, Plant
  PEPTIDE (1)..(3): Amino acid sequence motif
DRH

SEQ ID NO: 3 - DNA, 22 bp, Artificial Sequence
  Description of the artificial sequence Oligonucleotide-Primer
ctcgagaatggcttcctcaatg

SEQ ID NO: 4 - DNA, 29 bp, Artificial Sequence
  Description of the artificial sequence Oligonucleotide-Primer
gaattcccacacctgcatgcattgtactc

SEQ ID NO: 5 - DNA, 26 bp, Artificial Sequence
  Description of the artificial sequence Oligonucleotide-Primer
gaattccatgaactacttccgttatc

SEQ ID NO: 6 - DNA, 25 bp, Artificial Sequence
  Description of the artificial sequence Oligonucleotide-Primer
cccgggtcaaattacataatccgac